(19) United States
(12) Patent Application Publication
(10) Pub. No.: US 2011/0318740 A1
(43) Pub. Date: Dec. 29, 2011

(54) Reduced Interference from Single Strand Binding Proteins

(75) Inventors: Robert G. Eason, Los Gatos, CA (US); Timothy G. Geiser, San Mateo, CA (US); Vladimir I. Bashkirov, Davis, CA (US); Kristian M. Scaboo, Castro Valley, CA (US)

(73) Assignee: LIFE TECHNOLOGIES CORPORATION, Carlsbad, CA (US)

(21) Appl. No.: 13/149,697

(22) Filed: May 31, 2011

Related U.S. Application Data
(63) Continuation of application No. 11/609,058, filed on Dec. 11, 2006, now abandoned.
(60) Provisional application No. 60/748,999, filed on Dec. 9, 2005.

(51) Int. Cl. C12Q 1/68

(52) U.S. Cl. 435/6.11

(57) ABSTRACT

Disclosed, for example, are methods for detecting at least one target polynucleotide comprising cleaving a flap from a polynucleotide probe that is hybridized to a complementary target polynucleotide, and hybridizing the cleaved flap to a complementary capture probe immobilized on a surface, wherein said cleaving and/or hybridizing occurs in the presence of a single strand binding protein that is capable of binding single-stranded DNA, but that can bind neither the flap nor the capture probe. The cleaved flap and the complementary capture probe may each comprise PNA, RNA, LNA, L-DNA or other modified nucleotides or chimeras thereof that do not significantly bind to the single strand binding protein.

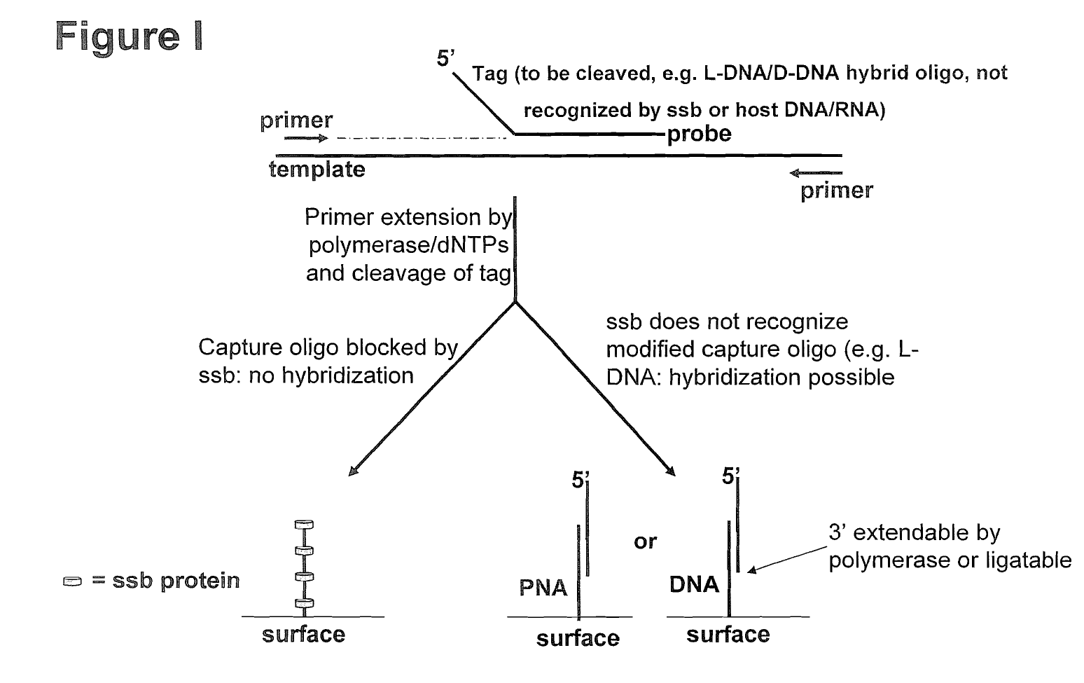

This application claims the benefit of priority of U.S. Provisional Application Ser. No. 60/748,999 filed Dec. 9, 2005, which is incorporated herein by reference.

The use of isothermal amplification methods in combination with oligo hybridization to capture oligonucleotides can be very beneficial. Capture oligos have many applications, among others in array technology (such as AB 1700 platform) or in our Electrochemical TaqMan methods, where a tag is cleaved during the TaqMan reaction and then captured to a surface immobilized capture oligonucleotide. In the presence of single strand binding proteins (ssb) the oligo-tag as well as the capture oligo can be bound to ssb and therefore prevent or reduce hybridization efficiency. SSB is used among others in an isothermal amplification method called RPA (recombinase polymerase amplification). This invention provides a way to overcome ssb blockage of the hybridization process and therefore helps to make RPA compatible to oligo capture.

In some aspects, the present disclosure provides modified oligonucleotides either as cleavage tags or as capture oligonucleotides or both. Modified cleavage tags or capture oligonucleotides can be used to prevent binding to ssb proteins (single strand binding proteins). Modified oligonucleotides can be PNA, RNA, LNA, L-DNA or other modified nucleotides or chimeras thereof This can decrease the binding efficiency towards single stranded binding proteins and can help prevent background from excess host DNA/RNA as well as inhibit inadvertent hybridization which can limit signal to background ratio. In the case of L-DNA capture probe, further background signal from amplification products or direct capture of host DNA or RNA can be reduced, since L-DNA does not hybridize to natural oligonucleotides, RNA and DNA. Also chimeric oligonucleotides of the modifications mentioned above with non-modified oligonucleotides are useful. The modified oligo part can hybridize, the non-modified part may be extended by polymerases or be ligated by ligases. Some examples and combinations are depicted in Figure I.

### Advantages

Run TaqMan assays or other assays isothermally or significantly reduce the number of cycles, thus increasing time to result
Prevent background signal from non-specific amplification such as amplification or capture products from host DNA or RNA
Be able to use large volumes of samples without the need of sample concentration
Be more sensitive for large volume samples
Add higher multiplexing capacity while maintaining high sensitivity and specificity
Reduce (or avoid) power consumption
Minimize sample prep due to reduced inhibition of enzymes
Simplify Devices
Produce simple and affordable handheld and portable devices and benchtop TaqMan instrumentation

### Exemplary Utilities

Handheld, portable/benchtop device for point of care use, pathogen detection, epidemiology and biosecurity surveillance.

1. A method for detecting at least one target polynucleotide comprising
cleaving a flap from a polynucleotide probe that is hybridized to a complementary target polynucleotide, and
hybridizing the cleaved flap to a complementary capture probe immobilized on a surface,
wherein said cleaving and/or hybridizing occurs in the presence of a single strand binding protein that is capable of binding single-stranded DNA, but that binds neither the flap nor the capture probe.

2. The method of claim 1, wherein said flap comprises a peptide nucleic acid sequence that is complementary to the capture probe.

3. The method of claim 1, wherein each of the cleaved flap and the complementary capture probe comprises PNA, RNA, LNA, L-DNA or other modified nucleotides or chimeras thereof.
